(19) United States
(12) Patent Application Publication
(10) Pub. No.: US 2021/0199610 A1
(43) Pub. Date: Jul. 1, 2021

(54) GAS SENSOR AND PACKAGING COMPONENT HAVING THE SAME

(71) Applicant: EPISTAR CORPORATION, Hsinchu (TW)

(72) Inventor: Wei-Chih PENG, Hsinchu (TW)

(21) Appl. No.: 16/836,779

(22) Filed: Mar. 31, 2020

(30) Foreign Application Priority Data
Dec. 27, 2019 (TW) 108217402

(51) Int. Cl. G01N 27/12; G01N 33/00

(52) U.S. Cl. CPC G01N 27/125; G01N 33/0016

(57) ABSTRACT

A gas sensor includes a substrate, an oxidization stack, a heating element, a sensing circuit, and a gas sensing element. The substrate has a first surface, a second surface opposite to the first surface, and a groove. The oxidization stack is on the substrate, and has a third surface and a fourth surface opposite to the third surface. The heating element is embedded in the oxidization stack. The sensing circuit is embedded in the oxidization stack. A distance between the sensing circuit and the fourth surface is shorter than a distance between the heating element and the fourth surface. The gas sensing element is located on the oxidization stack and coupled to the sensing circuit. The substrate and the oxidization stack collectively define a cavity substantially underneath the gas sensing element. The groove penetrates the substrate and extends outwardly from the cavity.

### CROSS-REFERENCE TO RELATED APPLICATION

This application claims priority to and the benefit of Taiwan Patent Application No. 108217402 filed on Dec. 27, 2019, which is incorporated by reference herein in its entirety.

### BACKGROUND

### Technical Field

This disclosure relates to a gas sensor and a packaging component having the same, in particular, to a microelectromechanical systems (MEMS) gas sensor and a packaging component having the same.

### Related Art

In general, a MEMS gas sensor has a sensing material on its top surface and a substrate on its rear surface. The operating temperature has to be high enough, so that the sensing material can easily react with the external gases for gas-sensing purpose. Therefore, a heating element is provided for heating the sensing material of the MEMS sensor. Moreover, in order to reduce the heating time, the substrate has a cavity located underneath the sensing material for reducing the heat generated by the heating element from being dissipated over the substrate. Hence, the temperature rising rate of the sensing material as well as the temperature of the sensing material can be further increased. The gas sensor can be used to detect external gases (such as carbon monoxide), so as to be applied in different ways, such as, environmental detections, house warnings, and industrial detections.

However, as realized by the inventor, in the packaging procedure of the MEMS gas sensor, the cavity of the substrate becomes a hermetic space when the substrate is bonded with the carrier plate of the packaging body. The gas pressure inside the cavity may be different from the gas pressure outside the substrate. The pressure difference in gas pressure may further cause the damage to the sensing material and affect the detection accuracy of the gas sensor. In one or some embodiments of the instant disclosure, a MEMS gas sensor structure is provided. Hence, in the packaging procedure, the possibility of failure of the sensing material due to the pressure difference between inside the cavity and outside the substrate can be reduced. Accordingly, the reliability, accuracy and sensitivity of gas sensor can be improved, and the structure of the packaging component can be effectively simplified so as to reduce the manufacturing cost.

### SUMMARY

In view of this, according to one or some embodiments of the instant disclosure, a MEMS gas sensor and a packaging component having the same are provided.

In one embodiment, a gas sensor includes a substrate, an oxidization stack, a heating element, a sensing circuit, and a gas sensing element. The substrate has a first surface, a second surface opposite to the first surface, and a groove. The oxidization stack is on the substrate, and has a third surface and a fourth surface opposite to the third surface. The heating element is embedded in the oxidization stack. The sensing circuit is embedded in the oxidization stack. A distance between the sensing circuit and the fourth surface is shorter than a distance between the heating element and the fourth surface. The gas sensing element is located on the oxidization stack and coupled to the sensing circuit. The substrate and the oxidization stack collectively define a cavity substantially underneath the gas sensing element. The groove penetrates the substrate and extends outwardly from the cavity.

In another embodiment, a gas sensor includes a substrate, an oxidization stack, a heating element, a sensing circuit, and a gas sensing element. The substrate has a first surface and a second surface opposite to the first surface. The oxidization stack is on the substrate, and has a third surface, a fourth surface opposite to the third surface. The oxidization stack faces the second surface with the third surface. The through hole extends from the third surface to the fourth surface. The heating element is embedded in the oxidization stack. The sensing circuit is embedded in the oxidization stack. A distance between the sensing circuit and the fourth surface is shorter than a distance between the heating element and the fourth surface. The gas sensing element is located on the oxidization stack and coupled to the sensing circuit. The substrate and the oxidization stack collectively define a cavity which resides right underneath the gas sensing element.

Detailed description of the characteristics and the advantages of the instant disclosure are shown in the following embodiments. The technical content and the implementation of the instant disclosure should be readily apparent to any person skilled in the art from the detailed description, and the purposes and the advantages of the instant disclosure should be readily understood by any person skilled in the art with reference to content, claims, and drawings in the instant disclosure.

### BRIEF DESCRIPTION OF THE DRAWINGS

The disclosure will become more fully understood from the detailed description given herein below for illustration only, and thus not limitative of the disclosure, wherein:

FIG. 1 illustrates a top view of a (microelectromechanical systems) MEMS gas sensor according to an exemplary embodiment of the instant disclosure;

FIG. 2 illustrates a cross-sectional view of the MEMS gas sensor along the XX′ direction shown in FIG. 1;

FIG. 3 illustrates a cross-sectional view of the MEMS gas sensor along the YY′ direction shown in FIG. 1;

FIG. 4 illustrates a cross-sectional view of an MEMS gas sensor according to another embodiment of the instant disclosure;

FIG. 5 illustrates a cross-sectional view of an MEMS gas sensor according to another embodiment of the instant disclosure;

FIG. 6 illustrates a cross-sectional view of an MEMS gas-sensing packaging component according to an exemplary embodiment of the instant disclosure;

FIG. 7 illustrates a top view of an MEMS gas sensor according to a further embodiment of the instant disclosure; and

FIG. 8 illustrates a cross-sectional view of the MEMS gas sensor along the AA′ direction shown in FIG. 7.

### DETAILED DESCRIPTION

Embodiments are provided, along with the figures, for facilitating the descriptions of the instant disclosure. It is understood that, a plenty of details are provided for readers to understand the disclosure; however, the inventions of the disclosure are still implementable in the premise that some or all of the details are omitted. In all the figures, same reference numbers designate identical or similar elements. It is worthy to mention that, the figures are provided for illustrative purposes, and are not used to indicate the actual size or number of the element. Moreover, some details may be omitted in the drawings for the sake of clarity for the drawings. It is understood that, the term “coupled to” may be directed to “directly electrical connection” or “indirectly electrical connection”.

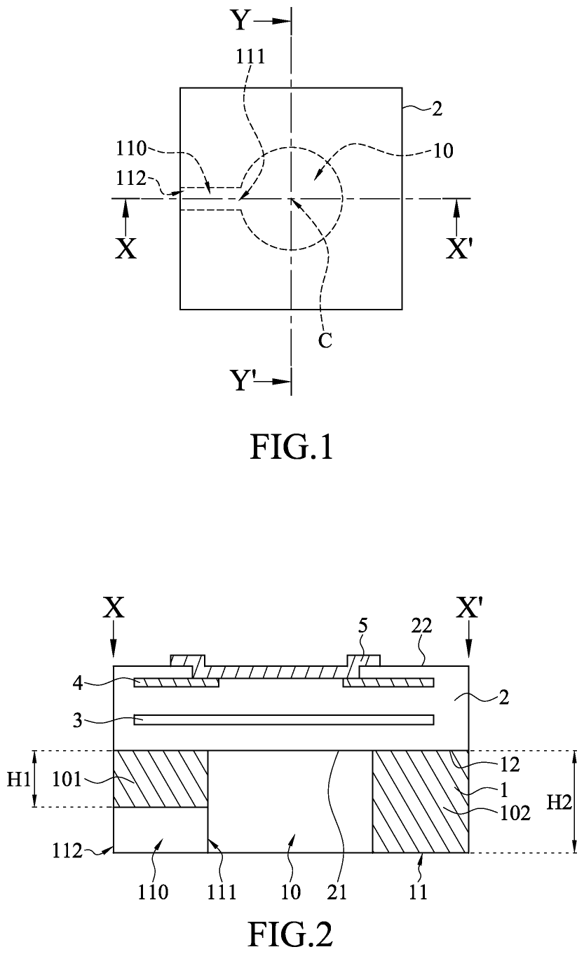

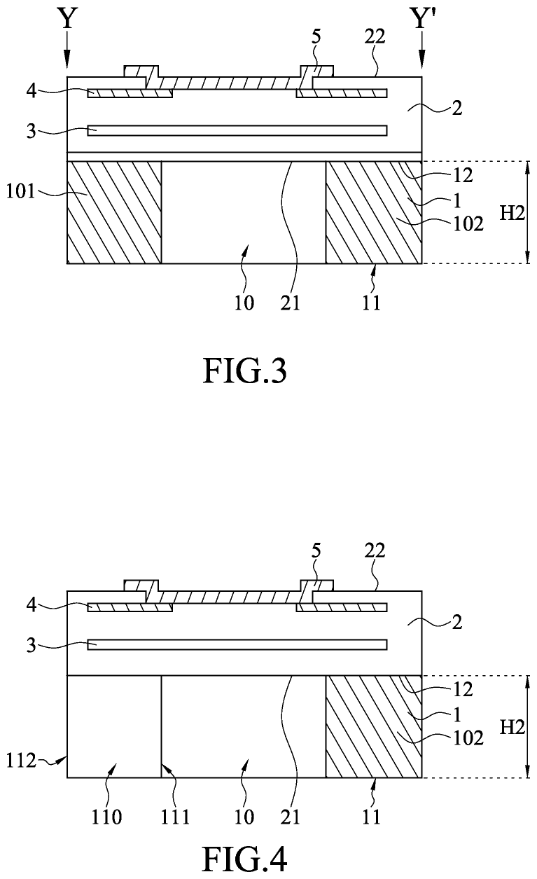

FIG. 1 illustrates a top view of a (microelectromechanical systems) MEMS gas sensor according to an exemplary embodiment of the instant disclosure. FIG. 2 illustrates a cross-sectional view of the MEMS gas sensor along line XX′ shown in FIG. 1. FIG. 3 illustrates a cross-sectional view of the MEMS gas sensor along line YY′ shown in FIG. 1. Please refer to FIGS. 1 to 3, the gas sensor comprises a substrate 1, an oxidization stack 2, a heating element 3, a sensing circuit 4, and a gas sensing element 5. With reference to FIGS. 1 and 2, in a top view, the gas sensing element 5 is substantially located at the center of the MEMS gas sensor. The substrate 1 has a cavity 10, a first surface 11, and a second surface 12. The first surface 11 is located in a place opposite to the second surface 12. The cavity 10 underneath the gas sensing element 5 is formed by completely or partially removing the substrate 1, in a manner of etching, or cutting and then etching. Moreover, in this embodiment, in a top view, the center of the cavity 10 and the center of the gas sensing element 5 are substantially overlapped with each other, and the area of the cavity 10 is greater than or equal to the area of the gas sensing element 5.

At the periphery of the substrate 1, a groove 110 is formed in a direction from the first surface 11 of the substrate 1 toward the second surface 12 of the substrate 1. The groove 110 extends outside the substrate 1 from the cavity 10. The gas pressure inside the cavity 10 and outside the substrate 1 can communicate with each other through the groove 110 to reach a balanced state or quasi-balanced state. The groove 110 may be formed by etching or by a manner of cutting and then etching. In one embodiment, a material of the substrate 1 may be, but not limited to, silicon, gallium arsenide, glass, or sapphire.

The oxidization stack 2 has a third surface 21 and a fourth surface 22, and the third surface 21 is opposite to the fourth surface 22. The third surface 21 faces to the substrate 1, and the oxidization stack 2 is formed on the second surface 12 of the substrate 1. For example, the oxidization stack 2 may be a multilayered oxide and formed on the substrate 1 through a thermal oxidation process to cover the cavity 10, but embodiments are not limited thereto. In one embodiment, the material of the oxidization stack 2 may be, but not limited to, silicon dioxide (SiO₂).

The heating element 3 is embedded in the oxidization stack 2, and the heating element 3 is near to the third surface 21 of the oxidization stack 2. In this embodiment, the heating element 3 may be a layer (or layers) of a metal material (or metal materials) so as to have a greater thermal conductivity. In another embodiment, the heating element 3 may be made of polycrystalline silicon, but embodiments are not limited thereto.

The sensing circuit 4 is embedded in the oxidization stack 2. A distance between the sensing circuit 4 and the fourth surface 22 of the oxidization stack 2 is shorter than a distance between the sensing circuit 4 and the third surface 21 of the oxidization stack 2. In this embodiment, the sensing circuit 4 may be made of a metal, which has a better conductivity. For example, the heating element 3 and the sensing circuit 4 may be formed by a deposition process with the same metal material.

The gas sensing element 5 is on the fourth surface 22 of the oxidization stack 2 and is coupled to the sensing circuit 4. The gas sensing element 5 may be made of, but not limited to, gold (Au), copper (Cu), silver (Ag), aluminum (Al), titanium (Ti), platinum (Pt), chromium (Cr), tantalum (Ta), molybdenum (Mo), tungsten (W), or the like. In one embodiment, the gas sensing element 5 is patterned through the photolithography process and is configured to detect certain gases, such as, but not limited to, carbon monoxide. An external control circuit (e.g., a MCU; not shown) can determine the existence and the concentration of the detected gases according to the change of the impedance of the gas sensing element 5 detected by the sensing circuit 4.

Accordingly, in one or some embodiments of the gas sensor, the heating element 3 heats the oxidization stack 2 for reaching the operating temperature of the gas sensing element 5, for example, the operating temperature of the gas sensing element 5 may be, but not limited to, in a range between 250 Celsius degrees and 350 Celsius degrees. Hence, the gas sensing element 5 is easily reacted with external gases at the operating temperature. Moreover, since the gas pressures inside the substrate 1 and outside the substrate 1 can communicate with each other through the groove 110, the gas pressures inside the cavity 10 and outside the substrate 1 can be a balanced state or quasi-balanced state. Hence, the problem as mentioned, i.e., the cavity of the substrate becomes a hermetic space when the substrate is bonded with the carrier plate of the packaging body in the subsequent packaging procedure, resulting in a pressure difference between inside the cavity and outside the substrate, thereby further causing the damage to the sensing material and affecting the detection accuracy of the gas sensor, can be avoided.

Please refer to FIGS. 1 and 2 again. In one embodiment, the cavity 10 of the substrate 1 is of a cylindrical shape in a top view. Moreover, from the top view of the gas sensor, the first end of the groove 110 directs toward the center of circle C of the cavity 10 so as to perform better heating and gas permeability performances. In one embodiment, the gas sensor is a cube with a length being about 1000 micrometers, where a distance between the circumference of the cylindrical-shaped cavity 10 and the edge of the substrate 1 is about 300 micrometers.

Please refer to FIG. 2. The first surface 11 of the substrate 1 may be processed by etching procedures to form the cavity 10 inside the substrate 1, wherein the opening of the cavity 10 faces the first surface 11. After the cavity 10 is formed in the substrate 1, the substrate 1 has a first side wall portion 101 and a second side wall portion 102 separated from the first side wall portion 101 by the cavity 10. In other words, the third surface 21 of the oxidization stack 2 is on the first side wall portion 101 and the second side wall portion 102, and the third surface 21 covers the cavity 10. As shown, the groove 110 is formed on one side of the cavity 10 and is defined through the first side wall portion 101 in a horizontal direction. In FIG. 2, the groove 110 is located below the first side wall portion 101 and is not defined through the first side wall portion 101 in a longitudinal direction, and the groove 110 extends from the cavity 10 inside the substrate 1 toward the outside of the substrate 1 to form a channel. Hence, the gas pressure inside the cavity 10 and the gas pressure outside the substrate 1 can be a balanced state or quasi-balanced state, and the gas sensing element 5 can be avoid getting failed or being interfered due to the pressure difference between inside and outside of the substrate. In this embodiment, along the XX′ direction of the substrate 1, only the first side wall portion 101 is penetrated by the groove 110, and the second side wall portion 102 is devoid of the groove 110. Hence, a height H1 of the first side wall portion 101 is less than a height H2 of the second side wall portion 102 in the XX′ direction of the substrate 1, as shown in FIG. 2. On the other hand, a depth of the first side wall portion 101 is less than that of the second side wall portion 102. As shown in FIG. 3, the substrate 1 is devoid of the groove 110 in the YY′ direction of the substrate 1. Hence, the first side wall portion 101 and the second side wall portion 102 has the same height H2 in the YY′ direction of the substrate 1.

Please refer to FIG. 4. In another embodiment, the groove 110 penetrates the first side wall portion 101 in the horizontal direction as well as in the longitudinal direction. In other words, the depth of the groove 110 is equal to the height H2 of the substrate 1. In this embodiment, the larger groove 110 facilitates the gas flowing and/or gas pressure releasing. Hence, the gas pressure inside the substrate 1 and the gas pressure outside the substrate 1 can be a balanced state or quasi-balanced state more rapidly.

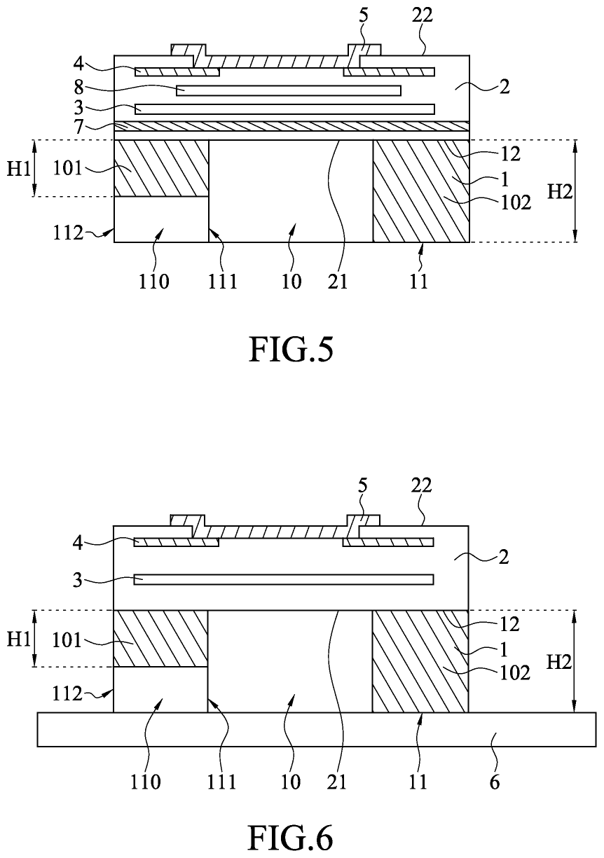

Please refer to FIG. 5. In yet another embodiment, the MEMS gas sensor further comprises a temperature sensing element 8. The temperature sensing element 8 is embedded in the oxidization stack 2, and the temperature sensing element 8 is between the heating element 3 and the sensing circuit 4 for monitoring the operating temperature. Hence, an external microcontroller unit (MCU) can adjust the heating element 3 according to the temperature detected by the temperature sensing element 8. The temperature sensing element 8 may be made of metal. For example, the heating element 3, the sensing circuit 4, and the temperature sensing element 8 may be formed by a deposition process with the same metal material.

In this embodiment, the MEMS gas sensor may further comprise a dielectric layer 7. The dielectric layer 7 is embedded in the oxidization stack 2, and the dielectric layer 7 is between the heating element 3 and the third surface 21 for improving the robustness of the oxidization stack 2. Hence, the reliability of the MEMS gas sensor can be enhanced. A material of the dielectric layer 7 may be, but not limited to, silicon nitride (SiNx).

Please refer to FIG. 6. A gas-sensing packaging component according to an exemplary embodiment of the present disclosure is illustrated. The gas-sensing packaging component comprises a gas sensor and a carrier plate 6. The gas sensor comprises a substrate 1, an oxidization stack 2, a heating element 3, a sensing circuit 4, and a gas sensing element 5. It is understood that, the elements within the gas sensor, features of the elements, the connection relationship between the elements, the functions of the elements, and varying embodiments of the elements are described as above and not repeated again herein.

The carrier plate 6 is located on the first surface 11 of the substrate 1 of the gas sensor. The substrate 1 has a cavity 10, and a first side wall portion 101 and a second side wall portion 102 which are arranged at two sides of the cavity 10. The carrier plate 6, the substrate 1, and the oxidization stack 2 collectively define a cavity 10 and a groove 110. The groove 110 is arranged between the first side wall portion 101 and the carrier plate 6, and is configured to communicate the cavity 10 inside the substrate 1 and the environment outside the substrate 1.

In another embodiment, the carrier plate 6 includes a control circuit. For example, the control circuit is, but not limited to, a microcontroller unit (MCU), combinational logic gates, logical operators, or the like.

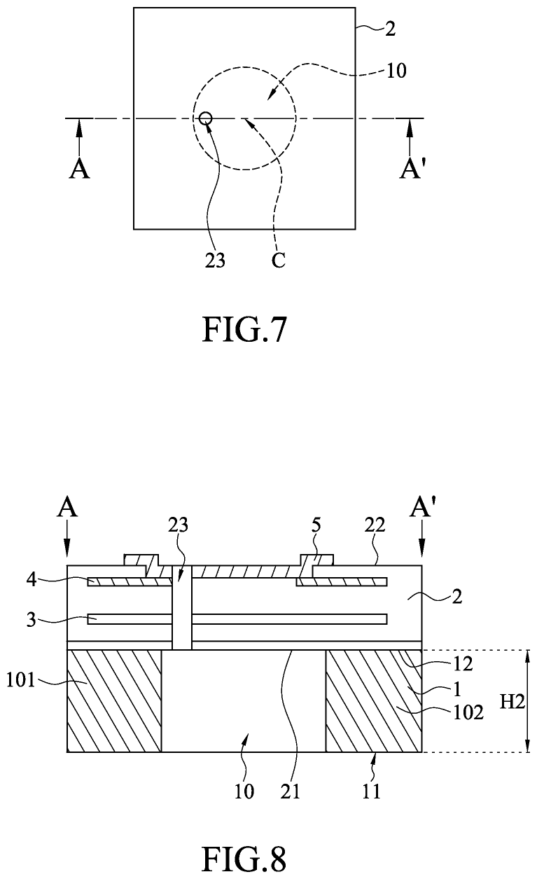

FIG. 7 illustrates a top view of an MEMS gas sensor according to a further embodiment of the instant disclosure. FIG. 8 illustrates a cross-sectional view of the MEMS gas sensor along the line AA′ shown in FIG. 7. Please refer to FIGS. 7 and 8. The gas sensor includes a substrate 1, an oxidization stack 2, a heating element 3, a sensing circuit 4, and a gas sensing element 5. In this embodiment, in the cross-sectional view as shown in FIG. 8, the gas sensing element 5 is substantially located at the center of the MEMS gas sensor. The substrate 1 has a cavity 10, a first surface 11, and a second surface 12 which is opposite to the first surface 11. A portion of the substrate 1 right beneath the gas sensing element 5 can be completely or partially removed, in a manner of etching or cutting and then etching, to form the cavity 10. Moreover, in a top view, the cavity 10 and the gas sensing element 5 are substantially overlapped with each other in or around their center portions, and the area of the cavity 10 is greater than or equal to the area of the gas sensing element 5.

The oxidization stack 2 has a third surface 21, a fourth surface 22, and a through hole 23. The third surface 21 is located in a position opposite to the fourth surface 22, and the through hole 23 penetrates the oxidization stack 2 and extends from the third surface 21 to the fourth surface 22. The oxidization stack 2 faces the substrate 1 with the third surface 21, and the oxidization stack 2 is on the second surface 12 of the substrate 1. For example, the oxidization stack 2 may be a multilayered oxide, and the oxidization stack 2 is formed on the substrate 1 through a thermal oxidation process to cover the cavity 10, but embodiments are not limited thereto. In one embodiment, the material of the oxidization stack 2 may be, but not limited to, silicon dioxide (SiO₂).

Please refer to FIG. 8. The difference between the embodiment shown in FIG. 2 and the embodiment shown in FIG. 8 is that, in this embodiment, the through hole 23 is formed on the oxidization stack 2 in a direction from the third surface 21 to the fourth surface 22. The through hole 23 extends outwardly from the cavity 10 to the topmost surface of the oxidization stack 2 to reach the environmental medium. In the embodiment shown in FIG. 8, the through hole 23 extends upwardly from the cavity 10, and two ends of the through hole 23 reach the third surface 21 and the fourth surface 22, respectively. In an exemplary embodiment, the through hole 23 may extend upwardly from the cavity 10 and turn at/round a right angle to extend in a horizontal direction. In other words, in this embodiment, two ends of the through hole 23 reach the third surface 21 and the outermost side surface of the gas sensor, but embodiments are not limited thereto. The cavity 10 and the environment outside the substrate 1 are in communication with each other through the through hole 23, so that the gas pressure inside the substrate 1 and the gas pressure outside the substrate 1 can be a balanced state or quasi-balanced state. The through hole 23 may be, but not limited to, formed by etching or by a manner of cutting and then etching. In some embodiments, the through hole 23 may be an opening with proper shape and size formed during the growth of the oxidization stack 2 in the photolithography process.

The heating element 3 is embedded in the oxidization stack 2 and close to the third surface 21 of the oxidization stack 2. In this embodiment, the heating element 3 may be a layer (or layers) of a metal material (or metal materials) so as to have a greater thermal conductivity. In another embodiment, the heating element 3 may be made of polycrystalline silicon, but embodiments are not limited thereto.

The sensing circuit 4 is embedded in the oxidization stack 2. A distance between the sensing circuit 4 and the fourth surface 22 of the oxidization stack 2 is shorter than a distance between the sensing circuit 4 and the third surface 21 of the oxidization stack 2. In this embodiment, the sensing circuit 4 may be made of metal for performing a better conductivity. For example, the heating element 3 and the sensing circuit 4 may be formed by a deposition process with the same metal material.

The gas sensing element 5 is located on the fourth surface 22 of the oxidization stack 2 and is coupled to the sensing circuit 4. The gas sensing element 5 may be made of, but not limited to, gold (Au), copper (Cu), silver (Ag), aluminum (Al), titanium (Ti), platinum (Pt), chromiume (Cr), tantalum (Ta), molybdenum (Mo), tungsten (W), or the like. In one embodiment, the gas sensing element 5 is patterned through the photolithography process and is configured to detect one or more gases, such as, but not limited to, carbon monoxide. An external control circuit (e.g., a MCU; not shown) can determine the existence and the concentration of the detected gases according to the change of the impedance of the gas sensing element 5 detected by the sensing circuit 4.

Accordingly, in one or some embodiments of the gas sensor, the heating element 3 heats the oxidization stack 2 for reaching the operating temperature of the gas sensing element 5, for example, the operating temperature of the gas sensing element 5 may be, but not limited to, in a range between 250 Celsius degrees and 350 Celsius degrees. Hence, the gas sensing element 5 is easily reacted with external gases at the operating temperature. Moreover, since the gas pressures inside the substrate 1 and outside the substrate 1 can communicate with each other through the through hole 110, the gas pressure inside the substrate 1 and the gas pressure outside the substrate 1 can be a balanced state or quasi-balanced state. Hence, the problem as mentioned, i.e., the cavity of the substrate becomes a hermetic space when the substrate is bonded with the carrier plate of the packaging body in the subsequent packaging procedure, resulting in a pressure difference inside the cavity and outside the substrate, thereby further causing the damage to the sensing material and affecting the detection accuracy of the gas sensor, can be avoided.

Please refer to FIGS. 7 and 8, in one embodiment, the cavity 10 of the substrate 1 is of a cylindrical shape so as to perform better heating and gas permeability performances. In one embodiment, the gas sensor is a cube with a length of about 1000 micrometers, where a distance between the circumference of the cylindrical-shaped cavity 10 and the edge of the substrate 1 is about 300 micrometers.

Accordingly, based on one or some embodiments of the instant disclosure, a gas sensor and a gas-sensing packaging component are provided. A gas exhausting channel is provided by forming the groove on the substrate and/or forming the through hole on the oxidization stack, so that the gas pressures inside the substrate and outside the substrate can be balanced, thereby the gas sensing material can be prevented from being failed due to the pressure difference between inside and outside the substrate. Accordingly, the reliability, accuracy and sensitivity for gas sensor can be improved, the structure of the packaging component can be effectively simplified, and the manufacturing costs can be reduced.

While the instant disclosure has been described by the way of example and in terms of the preferred embodiments, it is to be understood that the invention need not be limited to the disclosed embodiments. On the contrary, it is intended to cover various modifications and similar arrangements included within the spirit and scope of the appended claims, the scope of which should be accorded the broadest interpretation so as to encompass all such modifications and similar structures.

What is claimed is:

1. A gas sensor, comprising:
a substrate having a first surface, a second surface opposite to the first surface, and a groove;
an oxidization stack on the substrate, and having a third surface facing the substrate and a fourth surface opposite to the third surface, wherein the oxidization stack faces the second surface with the third surface;
a heating element embedded in the oxidization stack;
a sensing circuit embedded in the oxidization stack, wherein a distance between the sensing circuit and the fourth surface of the oxidization stack is shorter than a distance between the heating element and the fourth surface of the oxidization stack; and
a gas sensing element located on the oxidization stack and coupled to the sensing circuit,
wherein the substrate and the oxidization stack collectively define a cavity substantially underneath the gas sensing element, and
wherein the groove penetrates the substrate and extends outwardly from the cavity.

2. The gas sensor according to claim 1, wherein the substrate has a first side wall portion and a second side wall portion enclosing the cavity, the groove is at the first side wall portion, and a depth of the first side wall portion is less than that of the second side wall portion.

3. The gas sensor according to claim 1, wherein the cavity is of a cylindrical shape.

4. The gas sensor according to claim 1, further comprising a temperature sensing element between the heating element and the sensing circuit.

5. The gas sensor according to claim 1, further comprising a dielectric layer between the heating element and the third surface.

6. The gas sensor according to claim 5, wherein a material of the dielectric layer is silicon nitride.

7. The gas sensor according to claim 1, wherein a material of the substrate is silicon, gallium arsenide, glass, or sapphire.

8. The gas sensor according to claim 1, wherein the heating element is a metal layer.

9. A gas-sensing packaging component, comprising:
a gas sensor according to claim 1; and
a carrier plate disposed below the substrate.

10. The gas-sensing packaging component according to claim 9, wherein the carrier plate comprises a microcontroller unit (MCU).

11. A gas sensor, comprising:
a substrate having a first surface and a second surface opposite to the first surface;
an oxidization stack on the substrate and having a third surface, a fourth surface opposite to the third surface, wherein the oxidization stack faces the second surface with the third surface;
a through hole extending from the third surface to the fourth surface, a heating element embedded in the oxidization stack;
a sensing circuit embedded in the oxidization stack, wherein a distance between the sensing circuit and the fourth surface of the oxidization stack is shorter than a distance between the heating element and the fourth surface of the oxidization stack; and
a gas sensing element located on the oxidization stack and coupled to the sensing circuit,
wherein the substrate and the oxidization stack collectively define a cavity substantially underneath the gas sensing element.

12. The gas sensor according to claim 11, wherein the cavity is of a cylindrical shape.

13. The gas sensor according to claim 11, wherein a material of the oxidization stack is silicon dioxide.

14. The gas sensor according to claim 11, wherein the heating element is a metal layer.

15. A gas-sensing packaging component, comprising:
a gas sensor according to claim 11; and
a carrier plate disposed below the substrate.
